(19) United States
(12) Patent Application Publication
(10) Pub. No.: US 2025/0273344 A1
(43) Pub. Date: Aug. 28, 2025

(54) METHODS FOR THE DETECTION AND TREATMENT OF LUNG CANCER

(71) Applicant: Board of Regents, The University of Texas System, Austin, TX (US)

(72) Inventors: Samir HANASH, Houston, TX (US); Johannes F. FAHRMANN, Houston, TX (US); Ehsan IRAJIZAD, Houston, TX (US)

(21) Appl. No.: 19/206,866

(22) Filed: May 13, 2025

Related U.S. Application Data
(63) Continuation of application No. PCT/US2023/079957, filed on Nov. 16, 2023.
(60) Provisional application No. 63/384,188, filed on Nov. 17, 2022.

(51) Int. Cl. G16H 50/30; G01N 33/574; G16H 50/20

(52) U.S. Cl. CPC G16H 50/30; G01N 33/57423; G01N 33/57473; G16H 50/20

(57) ABSTRACT

A 3-marker metabolite panel (3MetP) comprising or consisting of diacetylspermine, arginine, and creatine riboside, optionally in combination with other panels and/or models, wherein the elevated levels and/or model score classify the patient as having lung cancer, for improved risk stratification for, diagnosis of, and treatment of lung cancer.

This application is a bypass continuation of International Application No. PCT/US2023/079957, filed Nov. 16, 2023, which claims the benefit of priority of U.S. Provisional Application No. 63/384,188, filed Nov. 17, 2022, the entirety of which is incorporated herein by reference.

This invention was made with government support under CA194733, CA213285, and CA086368 awarded by the National Institutes of Health. The government has certain rights in the invention.

Disclosed herein are methods and related kits for detection of lung cancer. Also provided are methods for treating a patient susceptible, or suspected of being susceptible, to lung cancer.

Lung cancer is the most prevalent cancer in the United States, with a five-year survival rate of less than 15%. Recently, therapies for lung cancer have begun to transition from a limited selection of radiation, folate metabolism, platinum-based drugs, and taxane-based drugs to more targeted treatments that require histological characterization of the tumor and/or the presence or absence of key biomarker or therapeutic target proteins.

Data from the National Lung Screening Trial (NLST) suggests that yearly screening of high-risk current and ex-smokers with thoracic low-dose computed tomography (LDCT) has been shown to reduce mortality due to lung cancer by 20%. In 2021, the United States Preventive Service Task Force (USPSTF) expanded the eligibility for LDCT screening and now recommends annual screening for lung cancer with LDCT for adults aged 50-80 years who have a smoking history greater than 20 pack-years and either currently smoke or have quit within the past 15 years. However, there are several negative aspects associated with CT screening in terms of morbidity, including over-diagnosis, false positives, over-treatment, and financial costs.

There is an abundance of literature on lung cancer risk prediction on the potential benefit of supplementing the USPSTF screening criteria with a risk-based model when identifying subjects for CT-screening. For instance, recently it was estimated that 20% of additional lung cancer deaths could be avoided by using a screening criterion based on individual risk assessment. The information required to utilize risk-prediction tools could be readily ascertained by a general practitioner—or potentially self-assessed using an online risk-calculator—making future lung cancer screening programs likely to implement such tools when assessing screening eligibility.

One such tool would be an individual-level risk-based screening criteria that accurately estimates the risk of lung cancer within the near future (e.g., 1-3 years) for a given subject. Several risk prediction models have been published that rely on demographic data (age, sex, etc.) and risk factor data from questionnaires, such as PLCOₘ₂₀₁₂ and the Liverpool Lung Project (LLP). Elevated levels of protein biomarkers have also been found to serve as useful predictors of the risk of developing lung cancer. A novel blood-based four-marker protein panel comprising or consisting of pro-surfactant protein B (pro-SFTPB), Mucin 16 (CA125), carcinoembryonic antigen (CEA), and cytokeratin-19 fragment (CYFRA21-1) is described in U.S. Ser. No. 16/484,177, the contents of which are hereby incorporated by reference in their entirety. The use of this panel in combination with PLCOₘ₂₀₁₂ has been found to significantly improve lung cancer risk assessment compared to former and current USPSFT criteria for lung cancer screening. As cellular and systemic metabolic adaptations occur from the earliest phases of cancer development, there is evidence that further improvements can be made through the identification and use of small molecule metabolites as cancer biomarkers.

Accordingly, a need exists for a method or test to aid the detection of lung cancer. A novel three-marker metabolite panel comprising or consisting of diacetylspermine (DAS), arginine, and creatine riboside has been discovered. In combination with the aforementioned four-marker protein panel and the PLCOₘ₂₀₁₂ risk prediction model, this model demonstrates superior lung cancer risk prediction in comparison to the four-marker protein panel, PLCOₘ₂₀₁₂, or the combination of both.

### SUMMARY

Provided herein is a method of treatment of lung cancer in a patient having elevated levels of, or an elevated risk score or positive risk profile based on the patient's measured levels of, diacetylspermine (DAS), arginine, and creatine riboside, and optionally, elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1), and optionally, an elevated PLCOₘ₂₀₁₂ model score, wherein the elevated levels, risk score, positive risk profile, and/or model score classify the patient as having lung cancer, comprising administering a therapeutically effective amount of a treatment for lung cancer to the patient.

Also provided is a method of treatment of lung cancer, comprising:
(a) identifying a patient having elevated levels of, or an elevated risk score or positive risk profile based on the patient's measured levels of, diacetylspermine (DAS), arginine, and creatine riboside, and optionally, elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1), and optionally, an elevated PLCOₘ₂₀₁₂ model score, wherein the elevated levels, risk score, positive risk profile, and/or model score classify the patient as having lung cancer; and
(b) administering a therapeutically effective amount of a treatment for lung cancer to the patient.

Also provided is a method of determining the risk of a subject for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the subject;
(b) optionally, calculating the PLCOₘ₂₀₁₂ model score of the subject; and
(c) classifying the subject as being at risk for lung cancer or not being at risk for lung cancer based on the measured levels or based on the measured levels and the model score.

Also provided is a method of producing a risk profile of a subject for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the subject;
(b) optionally, calculating the PLCOₘ₂₀₁₂ model score of the subject; and
(c) classifying the subject as being at risk for lung cancer or not being at risk for lung cancer based on the measured levels or based on the measured levels and the model score.

Also provided is a method of risk stratification for a patient at risk for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1 in a biological sample obtained from the patient;
(b) optionally, calculating the PLCOₘ₂₀₁₂ model score of the patient; and
(c) determining, by processor circuitry, the risk score for the patient, wherein the risk score is determined via a scoring function derived from metabolite profiles for biological samples, and optionally, PLCOₘ₂₀₁₂ model scores, taken from a plurality of individuals that were monitored for lung cancer.

Also provided is a method for calculating a patient's biomarker score or risk score for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the patient;
(b) optionally, calculating the PLCOₘ₂₀₁₂ model score of the patient; and
(c) calculating the biomarker score or risk score using the numerical values of the measured levels, and optionally, the PLCOₘ₂₀₁₂ model score, in a logistic regression model.

### BRIEF DESCRIPTION OF THE DRAWINGS

FIG. 1 depicts the predictive performance of the 3-marker metabolite panel (3MetP) for distinguishing case sera collected within 1 year of diagnosis compared to non-case sera in the Development Set.

FIG. 2 depicts the predictive performance of the 3-marker metabolite panel (3MetP) for distinguishing case sera collected within 1 year of diagnosis compared to non-case sera in the Testing Set.

### DETAILED DESCRIPTION

Provided herein is a method of treatment of lung cancer in a patient having elevated levels of, or an elevated risk score or positive risk profile based on the patient's measured levels of, diacetylspermine (DAS), arginine, and creatine riboside, and optionally, elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1), and optionally, an elevated PLCOₘ₂₀₁₂ model score, wherein the elevated levels, risk score, positive risk profile, and/or model score classify the patient as having lung cancer, comprising administering a therapeutically effective amount of a treatment for lung cancer to the patient.

Also provided is a method of treatment of lung cancer, comprising:
(a) identifying a patient having elevated levels of, or an elevated risk score or positive risk profile based on the patient's measured levels of, diacetylspermine (DAS), arginine, and creatine riboside, and optionally, elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1), and optionally, an elevated PLCOₘ₂₀₁₂ model score, wherein the elevated levels, risk score, positive risk profile, and/or model score classify the patient as having lung cancer; and
(b) administering a therapeutically effective amount of a treatment for lung cancer to the patient.

Also provided is a method of determining the risk of a subject for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the subject;
(b) optionally, calculating the PLCOₘ₂₀₁₂ model score of the subject; and
(c) classifying the subject as being at risk for lung cancer or not being at risk for lung cancer based on the measured levels or based on the measured levels and the model score.

Also provided is a method of producing a risk profile of a subject for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the subject;
(b) optionally, calculating the PLCOₘ₂₀₁₂ model score of the subject; and
(c) classifying the subject as being at risk for lung cancer or not being at risk for lung cancer based on the measured levels or based on the measured levels and the model score.

Also provided is a method of risk stratification for a patient at risk for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1 in a biological sample obtained from the patient;
(b) optionally, calculating the PLCOₘ₂₀₁₂ model score of the patient; and
(c) determining, by processor circuitry, the risk score for the patient, wherein the risk score is determined via a scoring function derived from metabolite profiles for biological samples, and optionally, PLCOₘ₂₀₁₂ model scores, taken from a plurality of individuals that were monitored for lung cancer.

Also provided is a method for calculating a patient's biomarker scores or risk score for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the patient;
(b) optionally, calculating the PLCOₘ₂₀₁₂ model score of the patient; and
(c) calculating the biomarker score or risk score using the numerical values of the measured levels, and optionally, the PLCOₘ₂₀₁₂ model score, in a logistic regression model.

In some embodiments, the biomarker scores or risk score for lung cancer are calculated with the equation: 0.420*[L-arginine]+0.383*[diacetylspermine]+0.184*[creatine riboside].

In some embodiments, the method further comprises calculating the PLCOₘ₂₀₁₂ model score.

In some embodiments, the method further comprises measuring the levels of or identifying a patient with elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1).

In some embodiments, calculating the PLCOₘ₂₀₁₂ model score comprises measuring a patient's age, ethnicity, educational level, body mass index (BMI), chronic obstructive pulmonary disease (COPD) status, personal history of cancer, family history of lung cancer, smoking status, smoking intensity, duration of smoking history, and duration after smoking cessation (i.e., quit time) and using the values to calculate a score with the PLCOₘ₂₀₁₂ logistic regression model.

In some embodiments, the levels of pro-SFTPB, CA125, CEA, and CYFRA21-1 are determined by an immunoassay.

In some embodiments, the level of DAS is determined by an immunoassay.

In some embodiments, the immunoassay is bead-based.

In some embodiments, the immunoassay is antibody-based.

In some embodiments, the measurements of age, ethnicity, educational level, body-mass index (BMI), chronic obstructive pulmonary disease (COPD) status, history of cancer, family history of lung cancer, smoking status, smoking intensity, duration of smoking history, and duration of smoking cessation (i.e., quit time) are determined by a patient survey.

In some embodiments, a combined model score is calculated using a logistic regression with the PLCOₘ₂₀₁₂ risk score and the biomarker score.

In some embodiments, the combined model score is calculated using the equation 0.8034*(0.420*[L-arginine]+0.383*[diacetylspermine]+0.184*[creatine riboside])+1.4238*(0.4730*[CA125]+06531*[CEA]+0.2612*[CYFRA21-1]+0.9238*[pro-SFTPB])+0.95*(PLCOₘ₂₀₁₂ model score).

In some embodiments, the lung cancer is early stage (e.g., stage I or II).

In some embodiments, the lung cancer is advanced (e.g., stage III or IV).

In some embodiments, the levels of diacetylspermine (DAS), arginine, and creatine riboside, and optionally, pro-surfactant protein B (pro-SFTPB), Mucin 16 (CA125), carcinoembryonic antigen (CEA), and cytokeratin-19 fragment (CYFRA21-1), are elevated relative to a reference patient or group that does not have lung cancer.

In some embodiments, the subject has a smoking history of ≥20 pack years.

In some embodiments, the subject is between the age of 50 and 80.

In some embodiments, each of diacetylspermine (DAS), arginine, and creatine riboside, and optionally, pro-surfactant protein B (pro-SFTPB), Mucin 16 (CA125), carcinoembryonic antigen (CEA), and cytokeratin-19 fragment (CYFRA21-1), generates a detectable signal.

In some embodiments, the detectable signals are detectable by a spectrometric method.

In some embodiments, the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

In some embodiments, the spectrometric method is mass spectrometry.

In some embodiments, the mass spectrometry is LC-TOF-MS.

In some embodiments, the treatment is chosen from surgery, chemotherapy, immunotherapy, radiation therapy, targeted therapy, or a combination thereof.

In some embodiments, the calculated biomarker score, risk score, model score, or risk profile is based on sensitivity and specificity values that correspond to the risk of the subject for lung cancer.

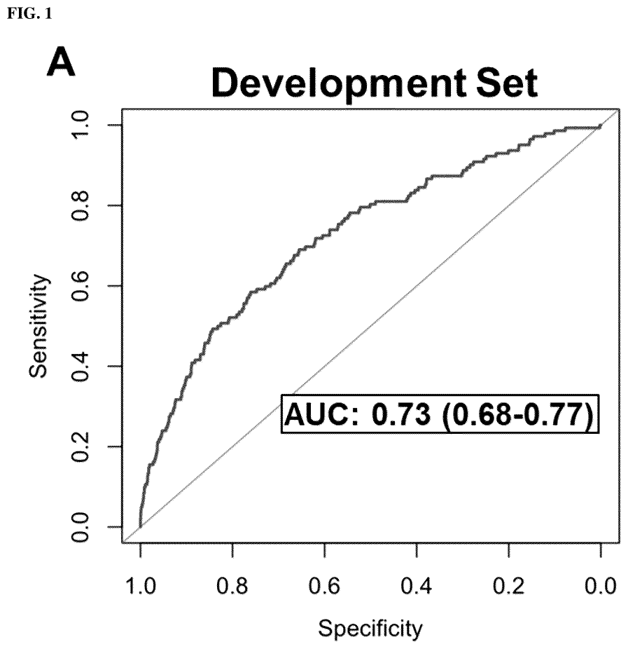

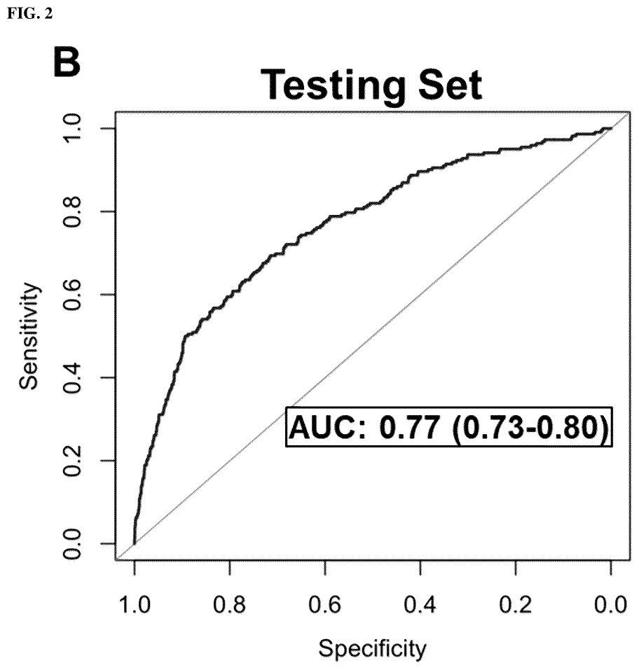

In some embodiments, the sensitivity and specificity values do not differ substantially from the curve in FIG. 1 or FIG. 2.

In some embodiments, the sensitivity and specificity values differ by less than 10%.

In some embodiments, the sensitivity and specificity values differ by less than 5%.

In some embodiments, the sensitivity and specificity values differ by less than 1%.

In some embodiments, the cutoff value comprises an AUC (95% CI) of at least 0.68.

In some embodiments, the method further comprises assigning the patient to an appropriate risk group based on the calculated risk score.

In some embodiments, there are at least two risk groups.

In some embodiments, the AUC of the method is greater than the AUC for a different biomarker, biomarkers, panel, assay, algorithm, model, or any combination thereof.

In some embodiments, the AUC is greater than 0.84.

In some embodiments, the AUC is between 0.84 and 0.89.

In some embodiments, the AUC is about 0.87.

In some embodiments, the sensitivity and specificity values at a ≥1.0%/6-year risk threshold of the method are greater than the sensitivity and specificity values for a different biomarker, biomarkers, panel, assay, algorithm, model or any combination thereof.

In some embodiments, the sensitivity is greater than 0.88 and the specificity is greater than 0.56.

In some embodiments, the sensitivity is between 0.88 and 0.90 and the specificity is between 0.56 and 0.60.

In some embodiments, the sensitivity is about 0.90 and the specificity is about 0.60.

In some embodiments, the model is PLCOₘ₂₀₁₂ alone.

In some embodiments, the biomarkers are pro-SFTPB, CA125, CEA, and CYFRA21-1 alone.

In some embodiments, the model is PLCOₘ₂₀₁₂ and the biomarkers are pro-SFTPB, CA125, CEA, and CYFRA21-1.

In some embodiments, the cutoff points of the respective methods are used for classification.

In some embodiments, the respective methods are analyzed by the same statistical methods.

In some embodiments, the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), are measured against a given threshold value or values.

In some embodiments, the values exceed the threshold value or values and the patient is classified as being at risk for lung cancer.

In some embodiments, the values are below the threshold value or values and the patient is classified as being not at risk for lung cancer.

In some embodiments, the patient is subsequently designated for further lung cancer screening or treatment.

In some embodiments, the screening is chosen from endoscopic ultrasound, magnetic resonance imaging (MRI), and computed topography (CT) scans.

In some embodiments, the screening is performed annually.

In some embodiments, the screening is performed semi-annually.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

As used herein, “lung cancer” refers to a malignant neoplasm of the lung characterized by the abnormal proliferation of cells, in which the growth of the cells exceeds and is uncoordinated with that of the normal tissues around it. In some embodiments, lung cancer may vary in severity, represented by stages I through IV. In some embodiments, lung cancer may be in an early stage (e.g., stage I or II), or it may be advanced (e.g., stage III or IV).

When a group is defined to be “null,” what is meant is that said group is absent.

As used herein, the terms “subject” or “patient” refer to a mammal, preferably a human, for whom a classification as lung cancer-positive or lung cancer-negative is desired, and for whom further treatment can be provided.

As used herein, a “reference patient,” “reference subject,” or “reference group” refers to a group of patients or subjects to which a test sample from a patient or subject suspected of having or being at risk for lung cancer may be compared. In some embodiments, such a comparison may be used to determine whether the test subject has lung cancer. A reference patient or group may serve as a control for testing or diagnostic purposes. As described herein, a reference patient or group may be a sample obtained from a single patient, or may represent a group of samples, such as a pooled group of samples.

As used herein, “healthy” refers to an individual in whom no evidence of lung cancer is found, i.e., the individual does not have lung cancer. Such an individual may be classified as “lung cancer-negative” or as having healthy lungs, or normal, non-compromised lung function. A healthy patient or subject has no symptoms of lung cancer, but may have benign lung nodules or masses, i.e., a combination of adenomas and cysts, or a non-cancerous lung condition or conditions, such as chronic obstructive pulmonary disease (COPD). In some embodiments, a healthy patient or subject may be used as a reference patient for comparison to diseased or suspected diseased samples for determination of lung cancer in a patient or a group of patients.

As used herein, “treating,” “treatment,” and the like means the administration of therapy to an individual who already manifests at least one symptom of a disease or condition or who has previously manifested at least one symptom of a disease or condition. For example, “treating” can include alleviating, abating, or ameliorating a disease or condition symptoms, preventing additional symptoms, ameliorating the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition. For example, the term “treating” in reference to a disorder means a reduction in severity of one or more symptoms associated with that particular disorder. Therefore, treating a disorder does not necessarily mean a reduction in severity of all symptoms associated with a disorder and does not necessarily mean a complete reduction in the severity of one or more symptoms associated with a disorder. As related to the present disclosure, the term may also mean the administration of pharmacological substances or formulations, or the performance of non-pharmacological methods including, but not limited to, radiation therapy and surgery. Pharmacological substances as used herein may include, but are not limited to, anticancer drugs including chemotherapeutics, polyamine inhibitors, hormone therapies, and targeted therapies. Examples of chemotherapeutics for lung cancer include paclitaxel/Taxol (e.g. albumin bound paclitaxel or nab-paclitaxel, trade name Abraxane®), erlotinib (Tarceva® and others), afatinib (Gilotrif®), gefitinib (Iressa®), bevacizumab (Avastin®), gemcitabine (Gemzar®), crizotinib (Xalkori®), ceritinib (Zykadia®), cisplatin/Platinol, carboplatin (Paraplatin®), docetaxel (Taxotere®), pemetrexed (Alimta®), and vinorelbine (Navelbine®); as well as combination regimens of chemotherapy including cisplatin+paclitaxel, TIP (paclitaxel/Taxol, ifosfamide, and cisplatin/Platinol), VeIP (vinblastine, ifosfamide, and cisplatin/Platinol), VIP (etoposide/VP-16, ifosfamide, and cisplatin/Platinol), VAC (vincristine, dactinomycin, and cyclophosphamide), and PEB (cisplatin/Platinol, etoposide, and bleomycin). The terms “pharmacological substance” and “anticancer therapy” may also include substances used in immunotherapy, such as checkpoint inhibitors. Treatment may include a multiplicity of pharmacological substances, or a multiplicity of treatment methods, including, but not limited to, surgery and chemotherapy.

As used herein, “amount” or “level” refers to a typically quantifiable measurement for a biomarker described herein, wherein the measurement enables comparison of the marker between samples and/or to control samples. In some embodiments, an amount or level is quantifiable and refers to the levels of a particular marker in a biological sample (e.g., blood, serum, urine, etc.), as determined by laboratory methods or tests such as an immunoassay, (e.g., antibodies), mass spectrometry, or liquid chromatography. In some embodiments, a marker may be present in the sample in an increased amount, or in a decreased amount. Marker comparisons may be based on direct measurement of the levels of a biomarker described herein, (e.g., through protein quantification or gene expression analysis) or may be based on measurement of e.g., reporter molecules, biomarker-receptor complexes, biomarker-relay-receptor complexes, or the like.

As used herein, the term “elevated” refers to a biomarker level or model score in a given subject that is greater relative to the same biomarker level or model score in a given set of healthy patients or subjects. In some embodiments, an elevated PLCOₘ₂₀₁₂ model score is 0.00948 or greater. In some embodiments, an elevated PLCOₘ₂₀₁₂ model score is 0.016082 or greater.

As used herein, the term “ELISA” refers to enzyme-linked immunosorbent assay. This assay generally involves contacting a fluorescently tagged sample of proteins with antibodies having specific affinity for those proteins. Detection of these proteins can be accomplished with a variety of means, including but not limited to laser fluorimetry.

As used herein, the term “regression” refers to a statistical method that can assign a predictive value for an underlying characteristic of a sample based on an observable trait (or set of observable traits) of said sample. In some embodiments, the characteristic is not directly observable. For example, the regression methods used herein can link a qualitative or quantitative outcome of a particular biomarker test, or set of biomarker tests, on a certain subject, to a probability that said subject is for lung cancer positive.

As used herein, the term “logistic regression” refers to a regression method in which the assignment of a prediction from the model can have one of several allowed discrete values. For example, the logistic regression models used herein can assign a prediction, for a certain subject, of either lung cancer-positive or lung cancer-negative.

As used herein, the term “biomarker score” refers to a numerical score for a given biomarker measured in a sample from a subject. The biomarker score is calculated by normalizing or weighting the measured level using a fixed coefficient as prescribed by the statistical method for a given biomarker panel. Biomarker scores are used as components in calculating a risk score for the subject. Elevated biomarker scores will carry more weight in risk score calculations and can indicate a higher risk for lung cancer for the subject.

As used herein, the term “risk score” refers to a single numerical value that indicates an asymptomatic human subject's risk for lung cancer as compared to the known prevalence of lung cancer in the disease cohort. The risk score is calculated through adding together the parameters of a statistical method derived from the subject for a given biomarker panel, which may take the form of biomarker scores, statistical model scores, or model constants. A higher risk score correlates to a higher risk for lung cancer in the subject. The risk score is empirically derived and will change depending on the data, cohort of the subject population, type of lung cancer, biomarkers chosen, occupational and environmental factors, and so on. In certain embodiments, the risk score as calculated for the human subject is the summation of the biomarker scores obtained from the subject. In certain embodiments, the risk score as calculated for the human subject is the summation of the biomarker scores obtained from the subject and one or more additional model constants. In certain embodiments, the risk score as calculated for a human subject is the summation of the biomarker scores obtained for the subject, normalized scores from one or more additional statistical models based on risk factors for the subject, and one or more additional model constants.

As used herein, the term “risk profile” refers to an assessment of a patient's risk score compared to those of a plurality of patients assessed using the same model, in which the patient is placed into an appropriate risk group based on a given score threshold. The score threshold is empirically derived and will change depending on the data, cohort of the subject population, type of lung cancer, biomarkers chosen, occupational and environmental factors, and so on. In certain embodiments, the patient's risk score exceeds the score threshold and their risk profile classifies them as being at risk for lung cancer (“positive”). In certain embodiments, the patient's risk profile is lower than the score threshold and classifies them as not being at risk for lung cancer (“negative”). In some embodiments, the score threshold is 0.005, or 0.5%, or greater. In some embodiments, the score threshold is 0.01, or 1%, or greater. In some embodiments, the score threshold is 0.05, or 5%, or greater. In some embodiments, the score threshold is 0.1, or 10%, or greater.

As used herein, the term “cutoff” or “cutoff point” refers to a mathematical value associated with a specific statistical method that can be used to assign a classification of lung cancer-positive of lung cancer-negative to a subject, based on said subject's biomarker score.

As used herein, when a numerical value above or below a cutoff value “is characteristic of lung cancer,” what is meant is that the subject, analysis of whose sample yielded the value, either has lung cancer or is at risk for lung cancer.

As used herein, the “use” of markers for diagnosing lung cancer refers to quantification of the levels or amounts in a biological sample of one or more markers described herein. Quantification may be done using any known methods or techniques in the art or described herein. In some embodiments, markers may be used or combined together as a panel for statistical comparison to other samples.

In some embodiments, using markers DAS, arginine, and creatine riboside together as a panel, or using markers DAS, arginine, creatine riboside, pro-SFTPB, CA125, CEA, CYFRA21-1, and the PLCOₘ₂₀₁₂ model score together as a panel may have an AUC (95% CI) of 0.55 or greater, including about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88. about 0.89, about 0.90, about 0.91, about 0.92, about 0.93, about 0.94, about 0.95, about 0.96, about 0.97, about 0.98, about 0.99, or the like.

In some embodiments, analyzing any of the marker panels described herein for diagnosis of lung cancer using fixed coefficients may result in an AUC (95% CI) of from about 0.55 to about 0.88 for distinguishing early-stage lung cancer, e.g., about 0.55, about 0.56, about 0.57, about 0.58, about 0.59, about 0.60, about 0.61, about 0.62, about 0.63, about 0.64, about 0.65, about 0.66, about 0.67, about 0.68, about 0.69, about 0.70, about 0.71, about 0.72, about 0.73, about 0.74, about 0.75, about 0.76, about 0.77, about 0.78, about 0.79, about 0.80, about 0.81, about 0.82, about 0.83, about 0.84, about 0.85, about 0.86, about 0.87, about 0.88, or the like. In some embodiments, analyzing these marker panels using fixed coefficients may result in an AUC (95% CI) of 0.86 for distinguishing early-stage lung cancer.

As used herein, a subject who is at “risk for lung cancer” is one who may not yet evidence overt symptoms of lung cancer, but who is producing levels of biomarkers which indicate that the subject has lung cancer or may develop it in the near term. A subject who has lung cancer or is suspected of harboring lung cancer may be treated for the cancer or suspected cancer.

As used herein, the term “classification” refers to the assignment of a subject as being at risk for lung cancer or not being at risk for lung cancer, based on the result of the biomarker score, risk score, or risk profile that is obtained for said subject.

As used herein, the term “Wilcoxon rank sum test,” also known as the Mann-Whitney U test, Mann-Whitney-Wilcoxon test, or Wilcoxon-Mann-Whitney test, refers to a specific statistical method used for comparison of two populations. For example, the test can be used herein to link an observable trait, in particular a biomarker level, to the absence or the risk for lung cancer in subjects of a certain population.

As used herein, the term “sensitivity” refers to, in the context of various biochemical assays, the ability of an assay to correctly identify those with a disease (i.e., the true positive rate). By comparison, as used herein, the term “specificity” refers to, in the context of various biochemical assays, the ability of an assay to correctly identify those without the disease (i.e., the true negative rate). Sensitivity and specificity are statistical measures of the performance of a binary classification test (i.e., classification function). Sensitivity quantifies the avoiding of false negatives, and specificity does the same for false positives.

As used herein, “fixed coefficients” or “fixed model coefficients” refers to a statistical method of standardizing coefficients in order to allow comparison of the relative importance of each coefficient in a regression model. In some embodiments, fixed coefficients involve using the same beta-coefficients from a logistic regression model to yield a risk score for the developed combination rule, which is ultimately used to make a clinical decision based on a decision threshold(s).

As used herein, a “sample” refers to a test substance to be tested for the presence of, and levels or concentrations thereof, of a biomarker as described herein. A sample may be any substance appropriate in accordance with the present disclosure, including, but not limited to, blood, blood serum, blood plasma, or any part thereof.

As used herein, a “metabolite” refers to small molecules that are intermediates and/or products of cellular metabolism. Metabolites may perform a variety of functions in a cell, for example, structural, signaling, stimulatory and/or inhibitory effects on enzymes. In some embodiments, a metabolite may be a non-protein, plasma-derived metabolite marker, such as including, but not limited to, DAS, arginine, and creatine riboside.

As used herein, the term “3-marker metabolite panel” or “3MetP” refers to a panel of three biomarkers, which includes DAS, arginine, and creatine riboside, useful for detecting lung cancer in a patient suspected of having lung cancer. In some embodiments, the 3-marker metabolite panel may be evaluated in combination with additional biomarkers or statistical models to enhance detection of lung cancer in biological samples from patients suspected of having lung cancer. Useful plasma protein biomarkers include, but are not limited to, pro-SFTPB, CA125, CEA, and CYFRA21-1. Useful statistical models include, but are not limited to, the PLCOₘ₂₀₁₂ and Liverpool Lung Project (LLP, LLPᵥ₂, or LLPᵥ₃) risk models.

As used herein, the term “ROC” refers to receiver operating characteristic, which is a graphical plot used herein to gauge the performance of a certain diagnostic method at various cutoff points. A ROC plot can be constructed from the fraction of true positives and false positives at various cutoff points.

As used herein, the term “AUC” refers to the area under the curve of the ROC plot. AUC can be used to estimate the predictive power of a certain diagnostic test. Generally, a larger AUC corresponds to increasing predictive power, with decreasing frequency of prediction errors. Possible values of AUC range from 0.5 to 1.0, with the latter value being characteristic of an error-free prediction method.

As used herein, the term “p-value” or “p” refers to the probability that the distributions of biomarker scores for lung cancer-positive and lung cancer-negative subjects are identical in the context of a Wilcoxon rank sum test. Generally, a p-value close to zero indicates that a particular statistical method will have high predictive power in classifying a subject.

As used herein, the term “CI” refers to a confidence interval, i.e., an interval in which a certain value can be predicted to lie with a certain level of confidence. As used herein, the term “95% CI” refers to an interval in which a certain value can be predicted to lie with a 95% level of confidence.

As used herein, the term “disease progression” or “early disease progression” is defined as upgrading of Gleason score and/or increased tumor volume on surveillance biopsy within 18 months after start of active surveillance.

The phrase “therapeutically effective” is intended to qualify the amount of active ingredients used in the treatment of a disease or disorder or on the effecting of a clinical endpoint.

### LIST OF ABBREVIATIONS

4MP=four-marker protein panel (pro-surfactant protein B (pro-SFTPB), Mucin 16 (CA125), carcinoembryonic antigen (CEA), and cytokeratin-19 fragment (CYFRA21-1)); AUC=area under the curve; DAS=N1, N12-diacetylspermine; HILIC=hydrophilic interaction liquid chromatography; HPLC=high performance liquid chromatography; NSCLC=Non-small-cell lung carcinoma; ROC=receiver operating characteristic; SEM=standard error of the mean; TCGA=The Cancer Genome Atlas; UPLC=ultra-high performance liquid chromatography; UPLC/MS=ultra-high performance liquid chromatography/mass spectrometry.

### EXAMPLES

The following examples are included to demonstrate embodiments of the disclosure. The following examples are presented only by way of illustration and to assist one of ordinary skill in using the disclosure. The examples are not intended in any way to otherwise limit the scope of the disclosure. Those of skill in the art should, in light of the present disclosure, appreciate that many changes can be made in the specific embodiments which are disclosed and still obtain a like or similar result without departing from the spirit and scope of the disclosure.

### Example 1: Specimen Sets

The PLCO Cancer Screening Trial was a randomized multicenter trial which aimed to evaluate the impact of screening for prostate, lung, colorectal and ovarian cancer on disease-specific mortality. All subjects involved in this study were enrolled with written consent to participate in the PLCO trial. Study recruitment and randomization began November 1993 and was completed in July 2001. Individual randomization to either the intervention or usual care group was within blocks that were stratified by screening center, sex, and age. PLCO eligibility criteria excluded subjects with a previous personal history of PLCO cancers, ongoing cancer treatment (excluding basal-cell and squamous-cell skin cancer), participation in another cancer screening or cancer primary prevention trial, and a recent screening test for prostate or colorectal cancer. The cohort comprises approximately 155,000 men and women aged 55 to 74 years old at baseline entry enrolled at ten screening centers across the United States. Participants randomized to the intervention group were offered a posterior-anterior chest radiograph at baseline and then annually for 3 more years. A chest radiograph was considered positive if a nodule, mass, infiltrate, or other abnormality considered suspicious for lung cancer was noted. Those with positive examination results were advised to seek diagnostic evaluation. In accordance with standard US practice, diagnostic evaluation was decided by the patients and their primary physicians, not by trial protocol. Study participants completed a baseline questionnaire at study entry that included demographic, personal, and medical information. Reporting of cancer status was based on annual questionnaires. Medical records were obtained to document diagnostic follow-up and characteristics of any diagnosed lung cancers.

A biorepository was created for blood specimens that were annually collected from consented, intervention group participants. There were 42,450 ever-smoker individuals in the intervention arm; 85% of the participants in the intervention arm had at least one collection. All histologically confirmed lung cancers from the ever-smoker subjects in the intervention arm that were diagnosed within two years of study entry (n=653 case participants) with pre-diagnostic sera available were selected for the current study. Non-case participants who have ever smoked were randomly selected (n=2,193 non-cases). Non-case participants were followed for an additional 13 years, during which they remained cancer-free. For each selected participant, all sera within two years of study entry, or up to the time of diagnosis for lung cancer cases, were included in the study specimen set (n=8,348 specimen total) (Table 1).

TABLE 1
Patient and Tumor characteristics for the PLCO Specimen Set

| | Development Set | | | | | Test Set | | | | | |
|---|---|---|---|---|---|---|---|---|---|---|---|
| | Cases^{a} | | Non-cases | | | Cases^{a} | | Non-cases | | | |
| | # of | # of | # of | | # of | # of | # of | # of | | # of | |
| Variable | Specimens | Participants | Specimens | | Participants | Specimens | Participants | Specimens | | Participants | |
| N | 267 | 259 | 3,764 | | 969 | 386 | 372 | 4,945 | | 1,224 | |
| Age, mean, SD | | 65 +/− 5 | | | 62 +/− 5 | | 64 +/− 5 | | | 62 +/− 5 | |
| Sex, N (%) | | | | | | | | | | | |
| Male | 183 | 177 | 2069 | | 534 | 248 | 240 | 2,201 | | 548 | |
| Female | 84 | 82 | 1695 | | 435 | 138 | 132 | 2,744 | | 676 | |
| Pack-Years (PY), N (%) | | | | | | | | | | | |
| <10 | 5 | 4 | 687 | | 173 | 15 | 14 | 879 | | 213 | |
| ≥10 to <20 | 21 | 21 | 689 | (23) | 174 (23) | 15 | 15 | 991 | (25) | 242 | (24) |
| ≥20 to <30 | 20 | 20 | 551 | (19) | 140 (18) | 40 | 39 | 708 | (18) | 174 | (18) |
| ≥30 | 219 | 212 | 1,698 | (58) | 446 (59) | 310 | 298 | 2,291 | (57) | 575 | (58) |

| Race, N (%) | | | | | | | | | | |
|---|---|---|---|---|---|---|---|---|---|---|
| White, Non-Hispanic | 223 | 217 | 2,987 | 763 | 347 | 334 | 4,766 | | 1,178 | |
| Black, Non-Hispanic | 25 | 24 | 326 | 90 | 34 | 33 | 118 | | 30 | |
| Hispanic | 8 | 8 | 118 | 30 | 5 | 5 | 38 | | 8 | |
| Asian | 10 | 9 | 264 | 67 | 0 (0) | 0 (0) | 11 | | 4 | (0.4) |
| Pacific Islander | 1 | 1 | 48 | 12 | 0 (0) | 0 (0) | 0 | (0) | 0 | (0) |

| | | | | | | | | | |
|---|---|---|---|---|---|---|---|---|---|
| American Indian | 0 | 0 | 21 | 7 | 0 (0) | 0 (0) | 12 | 4 | (0.4) |

| | | | | |
|---|---|---|---|---|
| Stage, N (%) | | | | |
| Stage I + II | 114 | 111 | 183 | 178 |
| Stage III + IV | 146 | 141 | 195 | 187 |
| Unknown | 7 | 7 | 8 | 7 |
| Histology, N (%) | | | | |
| Adeno-carcinoma | 102 | 99 | 150 | 142 |
| Squamous Cell Carcinoma | 63 | 58 | 76 | 76 |
| Small cell carcinoma | 36 | 36 | 58 | 55 |
| Other NSCLC | 66 | 68 | 84 | 81 |

Abbreviations: N, number;
SD, standard deviation;
PY, smoking pack years
^{a}Case specimen were limited to those specimens collected within two years of diagnosis.

### Example 2: Risk Model Based on Subject Characteristics

PLCOₘ₂₀₁₂ is a survey-based logistic-regression model that predicts the six-year risk of lung cancer diagnosis. This duration was chosen to optimize application and testing in the National Lung Screening Trial (NLST), which had a median six years of follow-up. Predictive variables in the PLCOₘ₂₀₁₂ model were obtained through baseline questionnaire information, and include age, race/ethnic group, education, body mass index, chronic obstructive pulmonary disease, personal history of cancer, family history of lung cancer and smoking status (current vs. former), intensity, duration, and quit time. The details of the PLCOₘ₂₀₁₂ model and its implementation have been described in Tammemägi et al., 2013, NEJM 368:728-736, which is hereby incorporated by reference in its entirety.

### Example 3: Metabolomic Analysis

Metabolomic assays are optimized to ensure that measurements for candidate metabolite biomarkers are above the limit-of-detection. Reference quality control samples and cohort specific pooled quality control samples were included in every batch to ensure data integrity. A total of 144 samples plus quality control samples were analyzed per analytical batch. All individuals who performed sample assays were blinded to specimen clinical information.

### Primary Metabolites

Serum metabolites were extracted from pre-aliquoted EDTA plasma (10 μL) with 30 μL of LCMS grade methanol (ThermoFisher) in a 96-well microplate (Eppendorf). Plates were heat sealed, vortexed for 5 min at 750 rpm, and centrifuged at 2000×g for 10 minutes at room temperature. The supernatant (10 μL) was carefully transferred to a 96-well plate, leaving behind the precipitated protein. The supernatant was further diluted with 10 μL of 100 mM ammonium formate, pH 3. For Hydrophilic Interaction Liquid Chromatography (HILIC) analysis, the samples were diluted with 60 μL LCMS grade acetonitrile (ThermoFisher). Each sample solution was transferred to 384-well microplate (Eppendorf) for LCMS analysis.

### Untargeted Analysis of Primary Metabolites

Untargeted metabolomics analysis was conducted on a Waters Acquity™ UPLC system with 2D column regeneration configuration (I-class and H-class) coupled to a Xevo G2-XS quadrupole time-of-flight (qTOF) mass spectrometer. Chromatographic separation was performed using a HILIC (Acquity™ UPLC BEH amide, 100 Å, 1.7 μm 2.1×100 mm, Waters Corporation, Milford, U.S.A) column at 45° C.

Quaternary solvent system mobile phases were (A) 0.1% formic acid in water, (B) 0.1% formic acid in acetonitrile and (D) 100 mM ammonium formate, pH 3. Samples were separated on the HILIC column using the following gradient profile: a starting gradient of 95% B and 5% D was increased linearly to 70% A, 25% B and 5% D over a 5 min period at a 0.4 mL/min flow rate, followed by a 1 min isocratic gradient at 100% A at a 0.4 mL/min flow rate.

A binary pump was used for column regeneration and equilibration. The solvent system mobile phases were (A1) 100 mM ammonium formate, pH 3, (A2) 0.1% formic in 2-propanol, and (B1) 0.1% formic acid in acetonitrile. The HILIC column was stripped using 90% A2 for 5 min followed by a 2 min equilibration using 100% B1 at a 0.3 mL/min flowrate.

### Mass Spectrometry Data Acquisition

Mass spectrometry data was acquired using the ‘sensitivity’ mode in positive electrospray ionization mode within 50-1200 Da range for primary metabolites. For the electrospray acquisition, the capillary voltage was set at 1.5 kV (positive), sample cone voltage 30V, source temperature at 120° C., cone gas flow 50 L/h and desolvation gas flow rate of 800 L/h with a scan time of 0.5 sec in continuum mode. Leucine Enkephalin; 556.2771 Da (positive) was used for lockspray correction and scans were performed at 0.5 min. The injection volume for each sample was 3 μL. The acquisition was carried out with instrument auto gain control to optimize instrument sensitivity over the sample acquisition time.

### Data Processing

Data were processed using Progenesis QI (Nonlinear, Waters). Peak picking and retention time alignment of LC-MS and MSe data were performed using the Progenesis QI software (Nonlinear, Waters). Data processing and peak annotations were performed using an in-house automated pipeline. Annotations were determined by matching accurate mass and retention times using customized libraries created from authentic standards and by matching experimental tandem mass spectrometry data against the NIST MSMS, LipidBlast or HMDB v3 theoretical fragmentations; for complex lipids retention time patterns characteristic of lipid subclasses was also considered. To correct for injection order drift, each feature was normalized using data from repeat injections of quality control samples collected every 10 injections throughout the run sequence. Measurement data were smoothed by Locally Weighted Scatterplot Smoothing (LOESS) signal correction (QC-RLSC) as previously described. Values are reported as ratios relative to the median of historical quality control reference samples run with every analytical batch for the given analyte. To account for any potential batch effects, metabolite readouts were auto-scaled, median-centered, and the values log 10 transformed.

### Assaying of the 4MP in the PLCO Specimen Set

Samples from all study participants for both training and testing, were sent on dry ice blinded to case-control status to the laboratory at MD Anderson Cancer Center, where they were kept below −80° C. until analysis. Concentrations for pro-SFTPB, CA125, CEA, and CYFRA21-1 were determined using bead-based immunoassays on the MAGPIX® instrument (Luminex Corporation, Austin TX). Samples were analyzed in batches of 36 samples in duplicates with matched cases and controls in the same batch in random order. Quality control procedures included 7 calibration standards, 2 Quality Control samples, and 1 blank sample run in duplicate in each batch. The coefficients of variation (CVs) within and between batches were 6.86% and 15.54% for CA125, 1.45% and 9.32% for CEA, 6.55% and 17.26% for pro-SFTPB, and 5.56% and 28.71% for CYFRA21-1, respectively. Biomarker scores for the 4MP were derived using fixed beta-coefficients from a previously developed logistic regression model (U.S. Ser. No. 16/484,177). Coefficients of variation (CV) values for pro-SFTPB, CA125, CEA, and CYFRA21-1 in quality control samples were 22.2, 12.8, 10.8, and 22.6 percent, respectively.

### Example 4: Feature Selection for Algorithm Training

A metabolomic screen was performed on a development set consisting of 267 sera collected within two years preceding diagnosis of lung cancer from 259 cases and 3,764 non-case sera (Table 1). A total of 75 uniquely annotated metabolites were quantified in all specimens. Of the quantified metabolites, 24, 32, and 31, were statistically significantly elevated (2-sided Wilcoxon Rank sum test FDR-adjusted p<0.05) in case sera collected between 0-6 months, 0-1 years, and 0-2 years preceding diagnosis compared to non-case sera (Table 2). Among the statistically significant metabolites were eight metabolites with previously reported relevance to cancer: acetylspermidine (AcSpmd), diacetylspermidine (DiAcSpmd), diacetylspermine (DAS), arginine (Arg), creatine riboside (CR), n-acetyllactosamine (NAcLac), 1-methyladenosine (1MA), and dimethylarginine (DMA). The predictive performance of the eight cancer-relevant metabolites increased the nearer the serum samples were taken to the time of diagnosis of lung cancer (Table 3).

TABLE 2
Performance estimates for individual metabolites quantified in the PLCO Development Set.

| Metabolite | AUC (95% CI) | | Pval | AUC (95% CI) | | Pval | AUC (95% CI) | | Pval |
|---|---|---|---|---|---|---|---|---|---|
| DAS | 0.707 | [0.654-0.759] | 0 | 0.71 | [0.667-0.752] | 0 | 0.66 | [0.626-0.694] | 0 |
| L-ARGININE | 0.677 | [0.617-0.737] | 0 | 0.666 | [0.616-0.717] | 0 | 0.635 | [0.596-0.673] | 0 |
| N-acetyllactosamine | 0.653 | [0.588-0.718] | 0.0002 | 0.666 | [0.615-0.717] | 0 | 0.636 | [0.598-0.674] | 0 |
| Choline | 0.649 | [0.583-0.714] | 0.0002 | 0.635 | [0.582-0.689] | 0 | 0.591 | [0.55-0.632] | 0 |
| N-acetyllactosamine | 0.631 | [0.565-0.697] | 0.001 | 0.629 | [0.577-0.682] | 0 | 0.611 | [0.572-0.649] | 0 |
| Acylcarnitine(C10:0) | 0.63 | [0.565-0.696] | 0.001 | 0.61 | [0.558-0.662] | 0.0002 | 0.58 | [0.541-0.62] | 0.0003 |
| Acylcarnitine(C18:1) | 0.619 | [0.55-0.688] | 0.003 | 0.625 | [0.571-0.679] | 0 | 0.579 | [0.538-0.62] | 0.0003 |
| Acylcarnitine(C14:0) | 0.616 | [0.549-0.683] | 0.004 | 0.619 | [0.567-0.67] | 0.0001 | 0.579 | [0.538-0.62] | 0.0003 |
| BETAINE; L-VALINE | 0.611 | [0.543-0.678] | 0.0056 | 0.601 | [0.548-0.654] | 0.0007 | 0.557 | [0.517-0.597] | 0.0117 |
| Plas_Lysophosphatidylcholine(P-18:0/0:0) or | 0.61 | [0.544-0.675] | 0.0058 | 0.632 | [0.578-0.685] | 0 | 0.585 | [0.543-0.627] | 0.0001 |
| Plas_Lysophosphatidylcholine(O-18:1) | | | | | | | | | |
| Prostaglandin A1 | 0.609 | [0.544-0.674] | 0.006 | 0.609 | [0.557-0.661] | 0.0003 | 0.577 | [0.538-0.617] | 0.0004 |
| Creatine riboside | 0.601 | [0.537-0.666] | 0.0116 | 0.616 | [0.566-0.666] | 0.0001 | 0.575 | [0.536-0.614] | 0.0006 |
| NG, NG-dimethyl-L-arginine | 0.598 | [0.531-0.665] | 0.0154 | 0.592 | [0.537-0.647] | 0.002 | 0.568 | [0.527-0.608] | 0.0025 |
| Acylcarnitine(C8:0) | 0.592 | [0.528-0.655] | 0.0243 | 0.554 | [0.504-0.605] | 0.0835 | 0.532 | [0.493-0.571] | 0.2008 |
| Indoleacetaldehyde | 0.592 | [0.514-0.671] | 0.0243 | 0.616 | [0.554-0.677] | 0.0001 | 0.613 | [0.569-0.657] | 0 |
| AcSpmd | 0.588 | [0.522-0.655] | 0.0315 | 0.575 | [0.523-0.627] | 0.0125 | 0.558 | [0.521-0.596] | 0.0105 |
| DEOXYCARNITINE | 0.585 | [0.518-0.651] | 0.0407 | 0.577 | [0.522-0.632] | 0.0113 | 0.534 | [0.493-0.575] | 0.167 |
| N-(3-acetamidopropyl)pyrrolidin-2-one | 0.583 | [0.525-0.641] | 0.0407 | 0.566 | [0.519-0.613] | 0.0306 | 0.56 | [0.524-0.597] | 0.0079 |
| DiAcSpmd | 0.583 | [0.516-0.65] | 0.0407 | 0.602 | [0.55-0.654] | 0.0007 | 0.594 | [0.556-0.631] | 0 |
| NG, NG-dimethyl-L-arginine | 0.576 | [0.507-0.645] | 0.0661 | 0.58 | [0.524-0.636] | 0.0082 | 0.554 | [0.512-0.595] | 0.0179 |
| Acylcarnitine(C16:0) | 0.575 | [0.505-0.646] | 0.0686 | 0.58 | [0.526-0.634] | 0.0082 | 0.555 | [0.514-0.596] | 0.0157 |
| Lysophosphatidylcholine(15:0) | 0.574 | [0.509-0.638] | 0.0752 | 0.595 | [0.547-0.644] | 0.0015 | 0.581 | [0.543-0.619] | 0.0003 |
| MALTOSE; MELIBIOSE; SUCROSE | 0.572 | [0.509-0.634] | 0.0834 | 0.582 | [0.534-0.63] | 0.0074 | 0.581 | [0.544-0.618] | 0.0003 |
| 1-METHYLADENOSINE | 0.571 | [0.496-0.646] | 0.0834 | 0.574 | [0.516-0.633] | 0.0139 | 0.561 | [0.52-0.602] | 0.0074 |
| Glycodeoxycholic acid | 0.565 | [0.499-0.63] | 0.1308 | 0.522 | [0.471-0.574] | 0.5334 | 0.511 | [0.473-0.549] | 0.6947 |
| THYROTROPIN RELEASING HORMONE | 0.563 | [0.502-0.623] | 0.1432 | 0.556 | [0.505-0.607] | 0.0769 | 0.535 | [0.495-0.574] | 0.1629 |
| Lysophosphatidylcholine(16:1) | 0.561 | [0.49-0.632] | 0.1568 | 0.6 | [0.545-0.655] | 0.0008 | 0.578 | [0.536-0.62] | 0.0004 |
| TMAO | 0.557 | [0.497-0.618] | 0.1905 | 0.522 | [0.47-0.573] | 0.5442 | 0.531 | [0.492-0.57] | 0.2015 |
| DEOXYCORTICOSTERONE ACETATE | 0.553 | [0.485-0.62] | 0.2419 | 0.569 | [0.514-0.624] | 0.0242 | 0.557 | [0.516-0.599] | 0.0117 |
| L-carnitine | 0.552 | [0.482-0.621] | 0.2524 | 0.578 | [0.526-0.63] | 0.0103 | 0.566 | [0.526-0.605] | 0.0036 |
| Acylcarnitine(C18:2n-6) | 0.544 | [0.477-0.61] | 0.3546 | 0.555 | [0.502-0.608] | 0.0827 | 0.54 | [0.499-0.58] | 0.0991 |
| ANILINE | 0.543 | [0.478-0.609] | 0.3546 | 0.547 | [0.494-0.6] | 0.1459 | 0.544 | [0.505-0.583] | 0.0589 |
| N-Undecanoylglycine | 0.541 | [0.48-0.602] | 0.3856 | 0.527 | [0.478-0.575] | 0.4487 | 0.498 | [0.461-0.536] | 0.9776 |
| L-GLUTAMIC ACID; N-METHYL-D- | 0.538 | [0.467-0.609] | 0.4201 | 0.566 | [0.511-0.621] | 0.0304 | 0.545 | [0.504-0.587] | 0.0501 |
| ASPARTIC ACID; Pyroglutamic acid | | | | | | | | | |
| 5′-METHYLTHIOADENOSINE | 0.537 | [0.473-0.601] | 0.4201 | 0.524 | [0.472-0.576] | 0.4958 | 0.519 | [0.48-0.557] | 0.4908 |
| Sphingosine | 0.537 | [0.471-0.604] | 0.4201 | 0.532 | [0.478-0.585] | 0.3742 | 0.532 | [0.492-0.571] | 0.2008 |
| Lysophosphatidylcholine(18:0) | 0.537 | [0.469-0.605] | 0.4201 | 0.574 | [0.522-0.626] | 0.0141 | 0.55 | [0.51-0.591] | 0.0267 |
| CORTISOL 21-ACETATE | 0.535 | [0.47-0.6] | 0.4507 | 0.546 | [0.492-0.599] | 0.1622 | 0.53 | [0.491-0.569] | 0.2232 |
| CAFFEINE | 0.533 | [0.466-0.601] | 0.4759 | 0.518 | [0.464-0.571] | 0.6179 | 0.53 | [0.489-0.571] | 0.2181 |
| NEPSILON.NEPSILON.NEPSILON- | 0.532 | [0.463-0.602] | 0.4843 | 0.529 | [0.475-0.584] | 0.419 | 0.52 | [0.48-0.56] | 0.4549 |
| TRIMETHYLLYSINE | | | | | | | | | |
| ALPHA-D-GLUCOSE; D-GALACTOSE; D- | 0.531 | [0.464-0.597] | 0.5083 | 0.538 | [0.487-0.589] | 0.2736 | 0.548 | [0.51-0.586] | 0.0364 |
| PSICOSE | | | | | | | | | |
| DEOXYCHOLATE | 0.529 | [0.458-0.6] | 0.5255 | 0.527 | [0.472-0.582] | 0.4487 | 0.514 | [0.473-0.554] | 0.6144 |
| PHOSPHO(ENOL)PYRUVIC ACID | 0.528 | [0.465-0.591] | 0.5353 | 0.536 | [0.488-0.584] | 0.2994 | 0.523 | [0.486-0.56] | 0.3944 |
| Lysophosphatidylcholine(20:2) | 0.523 | [0.457-0.588] | 0.6462 | 0.513 | [0.462-0.563] | 0.7209 | 0.504 | [0.466-0.541] | 0.9294 |
| 1-linoleoylglycerol | 0.522 | [0.454-0.59] | 0.6587 | 0.535 | [0.481-0.59] | 0.3133 | 0.538 | [0.497-0.579] | 0.1137 |
| L-PROLINE | 0.521 | [0.456-0.586] | 0.6687 | 0.517 | [0.466-0.568] | 0.6195 | 0.5 | [0.463-0.538] | 0.9904 |
| D-GLUCONO-1.5-LACTONE | 0.513 | [0.448-0.578] | 0.8051 | 0.52 | [0.469-0.571] | 0.5823 | 0.504 | [0.465-0.543] | 0.9294 |
| L-GLUTAMINE | 0.513 | [0.441-0.585] | 0.8051 | 0.53 | [0.475-0.585] | 0.4084 | 0.515 | [0.474-0.556] | 0.592 |
| Monoelaidin | 0.512 | [0.446-0.579] | 0.8082 | 0.54 | [0.488-0.592] | 0.2407 | 0.534 | [0.494-0.574] | 0.167 |
| CHOLATE | 0.511 | [0.441-0.581] | 0.8276 | 0.518 | [0.465-0.571] | 0.6179 | 0.497 | [0.458-0.536] | 0.955 |
| Pyroglutamic acid | 0.506 | [0.434-0.578] | 0.9132 | 0.527 | [0.471-0.582] | 0.4487 | 0.515 | [0.473-0.556] | 0.592 |
| L-PHENYLALANINE | 0.505 | [0.439-0.572] | 0.9186 | 0.509 | [0.455-0.562] | 0.7973 | 0.5 | [0.459-0.541] | 0.9904 |
| Lysophosphatidylcholine(17:0) | 0.504 | [0.431-0.577] | 0.9413 | 0.537 | [0.479-0.595] | 0.2868 | 0.504 | [0.461-0.547] | 0.9294 |
| Lysophosphatidylcholine(20:0) | 0.501 | [0.431-0.572] | 0.967 | 0.533 | [0.479-0.588] | 0.353 | 0.498 | [0.459-0.538] | 0.9776 |
| 1-Methylhistidine | 0.498 | [0.436-0.561] | 0.967 | 0.475 | [0.424-0.525] | 0.4667 | 0.481 | [0.443-0.519] | 0.4908 |
| cis-Quinceoxepane | 0.496 | [0.433-0.56] | 0.9413 | 0.488 | [0.436-0.539] | 0.7243 | 0.483 | [0.444-0.523] | 0.552 |
| L-HISTIDINE | 0.493 | [0.427-0.558] | 0.8996 | 0.472 | [0.418-0.526] | 0.4288 | 0.47 | [0.429-0.51] | 0.2181 |
| Sulfamethoxazole_exogenous | 0.491 | [0.428-0.554] | 0.8682 | 0.508 | [0.458-0.558] | 0.8047 | 0.491 | [0.454-0.527] | 0.7473 |
| cis-9, cis-12-Octadecadienoic acid; LINOLEATE | 0.486 | [0.416-0.557] | 0.8051 | 0.49 | [0.432-0.547] | 0.7568 | 0.495 | [0.453-0.537] | 0.9169 |
| Metformin | 0.484 | [0.421-0.548] | 0.7669 | 0.483 | [0.433-0.533] | 0.627 | 0.485 | [0.448-0.522] | 0.5838 |
| Chavicol O-beta-glucopyranoside | 0.483 | [0.414-0.552] | 0.7397 | 0.497 | [0.443-0.552] | 0.9308 | 0.484 | [0.444-0.523] | 0.5598 |
| Lysophosphatidylethanolamine(22:0) | 0.48 | [0.413-0.548] | 0.6862 | 0.489 | [0.436-0.543] | 0.7554 | 0.481 | [0.441-0.521] | 0.4908 |
| sterol | 0.472 | [0.407-0.537] | 0.5353 | 0.511 | [0.459-0.564] | 0.7523 | 0.501 | [0.462-0.54] | 0.983 |
| Lysophosphatidylethanolamine(16:0) | 0.469 | [0.398-0.541] | 0.5083 | 0.501 | [0.445-0.557] | 0.9629 | 0.514 | [0.472-0.555] | 0.6144 |
| Lysophosphatidylcholine(20:3); Lysophosphatidyl | 0.463 | [0.396-0.53] | 0.4201 | 0.471 | [0.417-0.525] | 0.423 | 0.488 | [0.447-0.528] | 0.6435 |
| choline(22:6) | | | | | | | | | |
| Lysophosphatidylethanolamine(18:2) | 0.46 | [0.39-0.531] | 0.4079 | 0.497 | [0.444-0.55] | 0.9298 | 0.513 | [0.473-0.553] | 0.6309 |
| BILIVERDIN | 0.456 | [0.39-0.522] | 0.3546 | 0.479 | [0.425-0.534] | 0.5579 | 0.479 | [0.439-0.519] | 0.4417 |
| 3-cis-Hydroxy-b,e-Caroten-3′-one | 0.451 | [0.384-0.518] | 0.2752 | 0.449 | [0.396-0.503] | 0.1142 | 0.468 | [0.429-0.508] | 0.2008 |
| Ceramide(32:1) | 0.449 | [0.385-0.513] | 0.2551 | 0.485 | [0.432-0.538] | 0.6629 | 0.478 | [0.439-0.517] | 0.4006 |
| Lysophosphatidylcholine(14:0) | 0.442 | [0.366-0.517] | 0.1796 | 0.495 | [0.436-0.554] | 0.8927 | 0.496 | [0.454-0.538] | 0.9294 |
| 3-Dehydrocarnitine | 0.417 | [0.364-0.47] | 0.0407 | 0.424 | [0.378-0.469] | 0.0114 | 0.445 | [0.407-0.484] | 0.016 |
| Lysophosphatidylcholine(18:2); Lysophosphatidyl | 0.416 | [0.349-0.484] | 0.0407 | 0.44 | [0.388-0.492] | 0.0517 | 0.471 | [0.432-0.51] | 0.2382 |
| choline(20:5) | | | | | | | | | |
| INDOLE-3-ACETALDEHYDE | 0.397 | [0.336-0.458] | 0.0104 | 0.407 | [0.356-0.457] | 0.0018 | 0.443 | [0.403-0.482] | 0.0117 |
| GUANOSINE | 0.378 | [0.317-0.438] | 0.0022 | 0.367 | [0.319-0.414] | 0 | 0.401 | [0.363-0.438] | 0 |
| piperine | 0.368 | [0.311-0.425] | 0.001 | 0.381 | [0.334-0.429] | 0.0001 | 0.39 | [0.354-0.426] | 0 |

TABLE 3
Time-dependent predictive performance of the 8 cancer-associated metabolites in the PLCO Development Set.

| | | | | | | | | Creatine |
|---|---|---|---|---|---|---|---|---|
| | 1MA | DMA | NAcLac | ARG | DAS | AcSpmd | DiAcSpmd | riboside |
| | 0-6 months | | | | | | | |
| AUC | 0.576 [0.511- | 0.611 [0.552- | 0.632 [0.574- | 0.681 [0.626- | 0.674 [0.617- | 0.603 [0.546- | 0.592 [0.53- | 0.676 [0.617- |
| | 0.642] | 0.671] | 0.69] | 0.736] | 0.731] | 0.66] | 0.653] | 0.734] |
| Sensitivity @ | 0.14 [0.07- | 0.14 [0.09- | 0.15 [0.1- | 0.16 [0.09- | 0.2 [0.14- | 0.07 [0.03- | 0.1 [0.04- | 0.2 [0.11- |
| 95% sp | 0.2] | 0.21] | 0.23] | 0.24] | 0.3] | 0.13] | 0.15] | 0.3] |
| Specificity @ | 0.08 [0.04- | 0.11 [0.03- | 0.08 [0.04- | 0.12 [0.07- | 0.14 [0.06- | 0.13 [0.09- | 0.09 [0.03- | 0.06 [0.03- |
| 95% sn | 0.14] | 0.19] | 0.3] | 0.33] | 0.32] | 0.19] | 0.19] | 0.24] |
| N0 | 3550 | 3550 | 3550 | 3550 | 3550 | 3550 | 3550 | 3550 |
| N1 | 91 | 91 | 91 | 91 | 91 | 91 | 91 | 91 |
| | 0-1 years | | | | | | | |
| AUC | 0.557 [0.505- | 0.586 [0.537- | 0.626 [0.579- | 0.667 [0.62- | 0.664 [0.618- | 0.569 [0.52- | 0.577 [0.53- | 0.648 [0.599- |
| | 0.609] | 0.634] | 0.674] | 0.714] | 0.71] | 0.617] | 0.627] | 0.696] |
| Sensitivity @ | 0.12 [0.07- | 0.12 [0.07- | 0.15 [0.11- | 0.19 [0.13- | 0.18 [0.12- | 0.07 [0.04- | 0.09 [0.04- | 0.15 [0.1- |
| 95% sp | 0.18] | 0.16] | 0.22] | 0.25] | 0.25] | 0.12] | 0.15] | 0.23] |
| Specificity @ | 0.07 [0.03- | 0.09 [0.07- | 0.09 [0.05- | 0.09 [0.06- | 0.13 [0.05- | 0.1 [0.08- | 0.08 [0.03- | 0.06 [0.03- |
| 95% sn | 0.12] | 0.17] | 0.25] | 0.13] | 0.28] | 0.14] | 0.11] | 0.16] |
| N0 | 3550 | 3550 | 3550 | 3550 | 3550 | 3550 | 3550 | 3550 |
| N1 | 142 | 142 | 142 | 142 | 142 | 142 | 142 | 142 |
| | 1-2 years | | | | | | | |
| AUC | 0.57 [0.515- | 0.584 [0.529- | 0.596 [0.539- | 0.655 [0.602- | 0.581 [0.527- | 0.551 [0.497- | 0.572 [0.52- | 0.581 [0.524- |
| | 0.626] | 0.64] | 0.653] | 0.707] | 0.635] | 0.606] | 0.625] | 0.637] |
| Sensitivity @ | 0.09 [0.05- | 0.12 [0.05- | 0.09 [0.05- | 0.13 [0.07- | 0.1 [0.06- | 0.07 [0.04- | 0.07 [0.03- | 0.07 [0.03- |
| 95% sp | 0.16] | 0.2] | 0.17] | 0.19] | 0.18] | 0.12] | 0.12] | 0.15] |
| Specificity @ | 0.12 [0.08- | 0.1 [0.07- | 0.07 [0.04- | 0.14 [0.02- | 0.11 [0.06- | 0.06 [0.02- | 0.09 [0.03- | 0.09 [0.01- |
| 95% sn | 0.15] | 0.16] | 0.16] | 0.26] | 0.22] | 0.13] | 0.16] | 0.17] |
| N0 | 3550 | 3550 | 3550 | 3550 | 3550 | 3550 | 3550 | 3550 |
| N1 | 107 | 107 | 107 | 107 | 107 | 107 | 107 | 107 |

### Example 5: Model Building and Testing

A combination rule was developed for distinguishing case sera collected within 1 year preceding a lung cancer diagnosis from non-case sera. Using Lasso regularization regression, a 3-marker metabolite panel (3MetP) consisting of DAS, arginine, and creatine riboside yielded an AUC of 0.73 (95% CI: 0.68-0.77) for case sera collected within one year of diagnosis compared to non-case sera (Tables 4-5; FIG. 1). The 3MetP had an AUC of 0.65 (95% CI: 0.60-0.70) for distinguishing case sera collected within 1-2 years of diagnosis compared to non-case sera (Table 5). Stratification of cases into early-stage (I+II) and late-stage (III-IV) lung cancer diagnosed within one year after blood draw compared with non-case sera resulted in AUCs of 0.64 (95% CI: 0.55-0.73) and 0.78 (95% CI: 0.73-0.84), respectively (Table 5). The performance of the 3MetP for distinguishing all case sera collected within one year of lung cancer diagnosis stratified into non-small cell lung cancer (NSCLC) or small cell lung cancer (SCLC) types compared with non-case sera was 0.72 (95% CI: 0.67-0.77) and 0.75 (95% CI: 0.63-0.87), respectively (Table 5).

Testing of the 3MetP was performed in an independent set consisting of 372 cases, from whom 386 sera were collected within 2 years of diagnosis along with 4,945 non-case sera. The 3MetP yielded respective AUCs of 0.77 (95% CI: 0.73-0.80) and 0.64 (95% CI: 0.58-0.69) for distinguishing case sera collected within 0-1 and 1-2 years of a lung cancer diagnosis compared to non-case sera (Table 6; FIG. 2). The performance of the 3MetP for early-stage (I+II) cases diagnosed within one year was 0.70 (95% CI: 0.65-0.75) and 0.82 (95% CI: 0.78-0.86) for advanced stage (III-IV) cases (Table 6). When considering sera collected within one year of a diagnosis of either NSCLC or SCLC compared to non-case sera, the 3MetP had resultant AUCs of 0.77 (95% CI: 0.73-0.80) and 0.77 (95% CI: 0.67-0.87) (Table 6).

TABLE 4
Model coefficients for the 3MetP.

| Metabolite | Coefficient |
|---|---|
| 1-methyladenosine | . |
| NG, NG-dimethyl-L-arginine | . |
| N-acetyllactosamine | . |
| L-arginine | 0.420 |
| Diacetylspermine (DAS) | 0.383 |
| Acetylspermidine (AcSpmd) | . |
| Diacetylspermidine (DiAcSpmd) | . |
| Creatine riboside | 0.184 |

TABLE 5
Performance estimates of the 3MetP in the PLCO Development Set.

| | | 3MetP | |
|---|---|---|---|
| | | 0-1 years | 1-2 years |
| All | AUC | 0.73 (0.68-0.77) | 0.65 (0.60-0.70) |
| | Sensitivity @ 95% sp | 0.23 (0.18-0.30) | 0.13 (0.07-0.21) |
| | Specificity @ 95% sn | 0.17 (0.12-0.28) | 0.11 (0.04-0.26) |
| | N0 | 3,550 | 3,550 |
| | N1 | 142 | 107 |
| Histology | AUC_Adeno | 0.71 (0.65-0.78) | 0.62 (0.53-0.71) |
| | AUC_Squamous | 0.79 (0.71-0.88) | 0.69 (0.59-0.79) |
| | AUC_NSCLC | 0.72 (0.67-0.77) | 0.64 (0.59-0.70) |
| | AUC_Small cell | 0.75 (0.63-0.87) | 0.69 (0.52-0.86) |
| Early | AUC_early | 0.64 (0.55-0.73) | 0.55 (0.45-0.64) |
| | Sensitivity @ 95% Specificity | 0.07 (0.00-0.19) | 0.06 (0.00-0.16) |
| | Specificity @ 95% Specificity | 0.15 (0.00-0.26) | 0.09 (0.04-0.27) |
| | N0_early | 3,550 | 3,550 |
| | N1_early | 42 | 31 |
| Late | AUC_late | 0.78 (0.73-0.84) | 0.71 (0.64-0.79) |
| | Sensitivity @ 95% Specificity | 0.34 (0.23-0.44) | 0.18 (0.09-0.29) |
| | Specificity @ 95% Specificity | 0.29 (0.17-0.49) | 0.14 (0.00-0.26) |
| | N0_late | 3,550 | 3,550 |
| | N1_late | 64 | 56 |

TABLE 6
Performance estimates of the 3MetP in the PLCO Test Set.

| | | 3MetP | |
|---|---|---|---|
| | | 0-1 years | 1-2 years |
| All | AUC | 0.77 (0.73-0.80) | 0.64 (0.60-0.69) |
| | Sensitivity @ 95% sp | 0.30 (0.23-0.36) | 0.16 (0.11-0.22) |
| | Specificity @ 95% sn | 0.24 (0.13-0.34) | 0.14 (0.07-0.19) |
| | N0 | 4,984 | 4,984 |
| | N1 | 222 | 169 |
| Histology | AUC_Adeno | 0.76 (0.71-0.81) | 0.63 (0.56-0.70) |
| | AUC_Squamous | 0.81 (0.75-0.87) | 0.63 (0.52-0.74) |
| | AUC_NSCLC | 0.77 (0.73-0.80) | 0.63 (0.58-0.68) |
| | AUC_Small cell | 0.77 (0.67-0.87) | 0.68 (0.57-0.78) |
| Early | AUC_early | 0.70 (0.65-0.75) | 0.58 (0.52-0.65) |
| | Sensitivity @ 95% Specificity | 0.18 (0.12-0.26) | 0.09 (0.05-0.18) |
| | Specificity @ 95% Specificity | 0.17 (0.07-0.32) | 0.11 (0.04-0.19) |
| | N0_early | 4,984 | 4,984 |
| | N1_early | 104 | 79 |
| Late | AUC_late | 0.82 (0.78-0.86) | 0.68 (0.62-0.75) |
| | Sensitivity @ 95% Specificity | 0.38 (0.27-0.47) | 0.21 (0.10-0.31) |
| | Specificity @ 95% Specificity | 0.31 (0.15-0.52) | 0.18 (0.10-0.23) |
| | N0_late | 4,984 | 4,984 |
| | N1_late | 101 | 77 |

### Contributions of the 3MetP for Improving Upon the Performance of a Combined 4MP Plus PLCOm2012 Model Among PLCO Participants Who Smoked ≥10 PY

The combination of the 4MP with the PLCOₘ₂₀₁₂ lung cancer risk model has been previously reported to improve predictive performance for identifying individuals with a smoking history of ≥10 PYs who would benefit from LDCT screening compared to either the PLCOₘ₂₀₁₂ model or USPSTF criteria (Fahrmann et al., 2022, Journal of Clinical Oncology, 40(8):876-883, which is hereby incorporated by reference in its entirety). To assess whether further improvements in lung cancer risk assessment could be achieved through the addition of the 3MetP, model scores derived from the 3MetP and the 4MP+PLCOₘ₂₀₁₂ were used to develop a logistic regression model for distinguishing case sera collected within one year of diagnosis from non-cases in the Training Set. The combined 3MetP+4MP+PLCOₘ₂₀₁₂ model yielded an AUC of 0.87 (95% CI: 0.84-0.89) for distinguishing case sera collected within one year of lung cancer diagnosis from non-case sera in the Test Set, which was improved compared to 4MP+PLCOₘ₂₀₁₂ alone (AUC: 0.85 (95% CI: 0.82-0.88); comparison P<0.001) (Tables 7-8).

TABLE 7
Performance estimates for the combined 3MetP + 4MP +
PLCOₘ₂₀₁₂ model and the 4MP + PLCOₘ₂₀₁₂ model in the PLCO
Development Set among individuals with a smoking history of ≥10PYs.

| | | 4MP + PLCOₘ₂₀₁₂ | 3MetP + 4MP + PLCOₘ₂₀₁₂ |
|---|---|---|---|
| All | AUC | 0.84 (0.81-0.88) | 0.86 (0.82-0.89) |
| | Sensitivity @ 95% sp | 0.47 (0.39-0.53) | 0.52 (0.44-0.61) |
| | Specificity @ 95% sn | 0.36 (0.24-0.49) | 0.39 (0.22-0.52) |
| | N0 | 2,726 | 2,726 |
| | N1 | 133 | 133 |
| Histology | AUC_Adeno | 0.83 (0.77-0.89) | 0.84 (0.78-0.90) |
| | AUC_Squamous | 0.83 (0.76-0.90) | 0.87 (0.80-0.94) |
| | AUC_NSCLC | 0.84 (0.80-0.88) | 0.85 (0.81-0.89) |
| | AUC_Small cell | 0.88 (0.80-0.97) | 0.90 (0.83-0.97) |
| Early | AUC_early | 0.81 (0.75-0.88) | 0.81 (0.75-0.88) |
| | Sensitivity @ 95% Specificity | 0.35 (0.22-0.51) | 0.35 (0.19-0.51) |
| | Specificity @ 95% Specificity | 0.39 (0.24-0.60) | 0.41 (0.37-0.50) |
| | N0_early | 2,726 | 2,726 |
| | N1_early | 37 | 37 |
| Late | AUC_late | 0.90 (0.86-0.94) | 0.92 (0.88-0.96) |
| | Sensitivity @ 95% Specificity | 0.60 (0.49-0.71) | 0.67 (0.57-0.79) |
| | Specificity @ 95% Specificity | 0.55 (0.35-0.79) | 0.66 (0.33-0.84) |
| | N0_late | 2,726 | 2,726 |
| | N1_late | 63 | 63 |

TABLE 8
Performance estimates for the combined 3MetP + 4MP +
PLCOₘ₂₀₁₂ model and the 4MP + PLCOₘ₂₀₁₂ model in the
PLCO Test Set among individuals with a smoking history of ≥10PYs.

| | | 4MP + PLCOₘ₂₀₁₂ | 3MetP + 4MP + PLCOₘ₂₀₁₂ |
|---|---|---|---|
| All | AUC | 0.85 (0.83-0.88) | 0.87 (0.84-0.89) |
| | Sensitivity @ 95% sp | 0.40 (0.32-0.49) | 0.43 (0.36-0.51) |
| | Specificity @ 95% sn | 0.41 (0.35-0.52) | 0.43 (0.37-0.58) |
| | N0 | 3,958 | 3,958 |
| | N1 | 208 | 208 |
| Histology | AUC_Adeno | 0.84 (0.80-0.89) | 0.86 (0.81-0.90) |
| | AUC_Squamous | 0.88 (0.84-0.91) | 0.90 [0.86-0.94) |
| | AUC_NSCLC | 0.85 (0.82-0.87) | 0.86 (0.84-0.89) |
| | AUC_Small cell | 0.874 (0.81-0.94) | 0.88 (0.82-0.94) |
| Early | AUC_early | 0.81 (0.76-0.85) | 0.82 (0.77-0.86) |
| | Sensitivity @ 95% Specificity | 0.33 (0.24-0.42) | 0.32 (0.21-0.42) |
| | Specificity @ 95% Specificity | 0.33 (0.20-0.50) | 0.36 (0.18-0.44) |
| | N0_early | 3,958 | 3,958 |
| | N1_early | 97 | 97 |
| Late | AUC_late | 0.90 (0.87-0.92) | 0.91 (0.89-0.94) |
| | Sensitivity @ 95% Specificity | 0.48 (0.38-0.59) | 0.53 (0.46-0.64) |
| | Specificity @ 95% Specificity | 0.56 (0.45-0.74) | 0.59 (0.52-0.74) |
| | N0_late | 3,958 | 3,958 |
| | N1_late | 94 | 94 |

To assess for potential clinical benefit, the sensitivity and specificity of the combined 3MetP+4MP+PLCOₘ₂₀₁₂ model was compared to that of the 4MP+PLCOₘ₂₀₁₂ model. At a ≥1.0%/6-year risk threshold, the combined 3MetP+4MP+PLCOₘ₂₀₁₂ model exhibited overall improved sensitivity (90% versus 88%) and improved specificity (60% versus 56%) compared to the combined 4MP+PLCOₘ₂₀₁₂ model (Tables 9-11).

If applied within the ever-smoker intervention arm 10+PY group, the combined 3Met+4MP+PLCOₘ₂₀₁₂ model would have identified an additional 14.3% lung cancer cases that would otherwise have been missed by the 4MP+PLCOₘ₂₀₁₂ model among the 119 cases who would otherwise receive a lung cancer diagnosis within a year, as well as 1,184 (8.4%) fewer non-cases among 14,122 otherwise referred for annual screening (Table 9). Based on the ≥1.0%/6-year threshold, 108 of the 119 participants who received a lung cancer diagnosis within one year were criteria positive with the combined 3MetP+4MP+PLCOₘ₂₀₁₂ model (Table 9).

TABLE 9
Accuracy performances in the Test Set for the 4MP + PLCOₘ₂₀₁₂ model
and the combined 3MetP + 4MP + PLCOₘ₂₀₁₂ model at a fixed ≥1.0%
6-year risk threshold to be comparable to USPSTF2021 criteria in ESIA10+.

| | ≥1.0% Risk Threshold | | | | | |
|---|---|---|---|---|---|---|
| | | | 1-year | | 1-year | |
| Criteria | N1^{d} | N0 | Sensitivity^{e} | Specificity | TP^{f} | Fp^{f} |
| USPSTF2021^{a} | 119 | 32,243 | 0.79 | 0.49 | 94 | 16,356 |
| Combined | 119 | 32,243 | 0.88 | 0.56 | 105 | 14,122 |
| 4MP + PLCOₘ₂₀₁₂ Model^{b} | | | | | | |
| Combined | 119 | 32,243 | 0.90 | 0.60 | 108 | 12,889 |
| Met3P + 4MP + PLCOₘ₂₀₁₂ Model^{c} | | | | | | |

^{a}Performance of the USPSTF eligibility criteria was calculated directly from the ESIA10+.
^{b}As published in Fahrmann et al; not re-calibrated.
cCalibrated to risk in the ESIA10+.
^{d}Participant counts (N0- non-cases; N1- cases). Total number of cases diagnosed within 1 year based on the total number of cases in ESIA10+ over the 6-year trial period.
^{e}1-year Sensitivity is the proportion of positive test results among participants who would, in the absence of screening, be diagnosed with lung cancer within one year.
^{f}The number of true positives (TP), positive test results among participants within 1 year of a lung cancer diagnosis, and false positives (FP), positive test results among non-cases, if the tests were applied to the ESIA10+.

TABLE 10
Strata specific performances in the Development Set
for the 4MP, PLCOₘ₂₀₁₂, 3MetP, 4MP + PLCOM₂₀₁₂, and
3MetP + 4MP + PLCOM₂₀₁₂ models at 6-year
risk thresholds of ≥1.0% in ESIA10+.

| | | | 1-year | | 1-year | |
|---|---|---|---|---|---|---|
| Strata^{a} | N1^{b} | N0 | Sensitivity^{c} | Specificity | TP^{d} | FP^{d} |
| | | | ≥1.0% Risk Threshold | | | |
| | | | 4-Marker Panel (4MP)^{e} | | | |
| Low^{h} | 10 | 10,967 | 0.353 | 0.885 | 4 | 1,256 |
| Medium | 24 | 7,215 | 0.905 | 0.347 | 21 | 4,712 |
| High | 85 | 14,061 | 0.946 | 0.208 | 81 | 11,137 |
| | | | 3MetP | | | |
| Low^{h} | 10 | 10,967 | 0.375 | 0.908 | 4 | 1,009 |
| Medium | 24 | 7,215 | 0.850 | 0.275 | 20 | 5,231 |
| High | 85 | 14,061 | 0.990 | 0.026 | 84 | 13,695 |
| | | | PLCOₘ₂₀₁₂^{f} | | | |
| Low^{h} | 10 | 10,967 | 0.250 | 0.915 | 3 | 935 |
| Medium | 24 | 7,215 | 0.850 | 0.441 | 20 | 4,030 |
| High | 85 | 14,061 | 0.972 | 0.149 | 83 | 11,960 |
| | | | Combined 4MP + PLCOₘ₂₀₁₂ Model^{g} | | | |
| Low^{h} | 10 | 10,967 | 0.312 | 0.910 | 3 | 988 |
| Medium | 24 | 7,215 | 0.800 | 0.600 | 19 | 2,888 |
| High | 85 | 14,061 | 0.981 | 0.311 | 84 | 9,686 |
| | | | Combined 3MetP + 4MP + PLCOₘ₂₀₁₂ Model | | | |
| Low^{h} | 10 | 10,967 | 0.400 | 0.901 | 4 | 1,086 |
| Medium | 24 | 7,215 | 0.737 | 0.646 | 18 | 2,554 |
| High | 85 | 14,061 | 0.947 | 0.369 | 80 | 8,872 |

^{a}Strata according to eligibility under USPSTF2021 recommendations. Low: 10-20 smoking packs per year or 20-29 smoking packs per year and smoking quit time ≥15 years; Medium- 20-29 smoking pack years and smoking quit time <15 years or 30+ smoking packs per years and smoking quit time ≥15 years; High- 30+ smoking packs per year and smoking quit time <15 years.
^{b}Participant counts (N0- non-cases; N1- cases). Total number of cases diagnosed within 1 year based on the total number of cases in ESIA10+ over the 6-year trial period.
^{c}1-year Sensitivity is the proportion of positive test results among participants who would, in the absence of screening, be diagnosed with lung cancer within one year.
^{d}The number of true positives (TP), positive test results among participants within 1 year of a lung cancer diagnosis, and false positives (FP), positive test results among non-cases, if the tests were applied to the ESIA10+.
^{e}Calibrated by strata to risk in the ESIA10+.
^{f}As published in Tammemägi et al; not recalibrated.
^{g}Calibrated to risk in the ESIA10+.
^{h}Quantities reported for low, medium, and high-risk strata are estimated from the specimen set. Quantities reported for the combined strata (Total) are weighted to reflect the distribution of low, medium, and high-risk participants.

TABLE 11
Strata specific performances in the Test Set for
the 4MP, PLCOₘ₂₀₁₂, 3MetP, 4MP + PLCOM₂₀₁₂,
and 3MetP + 4MP + PLCOM₂₀₁₂ models at
6-year risk thresholds of ≥1.0% in ESIA10+.

| | | | 1-year | | 1-year | |
|---|---|---|---|---|---|---|
| Strata^{a} | N1^{b} | N0 | Sensitivity^{c} | Specificity | TP^{d} | FP^{d} |
| | | | ≥1.0% Risk Threshold | | | |
| | | | 4-Marker Panel (4MP)^{e} | | | |
| Low^{h} | 10 | 10,967 | 0.438 | 0.887 | 4 | 1,245 |
| Medium | 24 | 7,215 | 0.864 | 0.353 | 20 | 4,668 |
| High | 85 | 14,061 | 0.986 | 0.169 | 84 | 11,679 |
| | | | 3MetP | | | |
| Low^{h} | 10 | 10,967 | 0.312 | 0.934 | 3 | 722 |
| Medium | 24 | 7,215 | 0.909 | 0.296 | 22 | 5,081 |
| High | 85 | 14,061 | 0.987 | 0.026 | 84 | 13,699 |
| | | | PLCOₘ₂₀₁₂^{f} | | | |
| Low^{h} | 10 | 10,967 | 0.438 | 0.897 | 4 | 1,135 |
| Medium | 24 | 7,215 | 0.841 | 0.503 | 20 | 3,588 |
| High | 85 | 14,061 | 1.000 | 0.111 | 85 | 12,501 |
| | | | Combined 4MP + PLCOₘ₂₀₁₂ Model^{g} | | | |
| Low^{h} | 10 | 10,967 | 0.500 | 0.906 | 5 | 1,031 |
| Medium | 24 | 7,215 | 0.682 | 0.611 | 16 | 2,807 |
| High | 85 | 14,061 | 0.986 | 0.269 | 84 | 10,274 |
| | | | Combined 3MetP + 4MP + PLCOₘ₂₀₁₂ Model | | | |
| Low^{h} | 10 | 10,967 | 0.500 | 0.909 | 5 | 999 |
| Medium | 24 | 7,215 | 0.773 | 0.657 | 18 | 2,477 |
| High | 85 | 14,061 | 0.980 | 0.319 | 83 | 9,574 |

^{a}Strata according to eligibility under USPSTF2021 recommendations. Low: 10-20 smoking packs per year or 20-29 smoking packs per year and smoking quit time ≥15 years; Medium- 20-29 smoking pack years and smoking quit time <15 years or 30+ smoking packs per years and smoking quit time ≥15 years; High- 30+ smoking packs per year and smoking quit time <15 years.
^{b}Participant counts (N0- non-cases; N1- cases). Total number of cases diagnosed within 1 year based on the total number of cases in ESIA10+ over the 6-year trial period.
^{c}1-year Sensitivity is the proportion of positive test results among participants who would, in the absence of screening, be diagnosed with lung cancer within one year.
^{d}The number of true positives (TP), positive test results among participants within 1 year of a lung cancer diagnosis, and false positives (FP), positive test results among non-cases, if the tests were applied to the ESIA10+.
^{e}Calibrated by strata to risk in the ESIA10+.
^{f}As published in Tammemägi et al; not recalibrated.
^{g}Calibrated to risk in the ESIA10+.
^{h}Quantities reported for low, medium, and high-risk strata are estimated from the specimen set. Quantities reported for the combined strata (Total) are weighted to reflect the distribution of low, medium, and high-risk participants.

The number of criteria positive non-cases that would need to be referred to screening to refer one participant who would otherwise receive a lung cancer diagnosis among different risk strata is shown in Table 12. For the 3MetP+4MP+PLCOₘ₂₀₁₂ model, 120 criteria positive non-cases would need to be referred to identify one case, compared to 135 for the 4MP+PLCOₘ₂₀₁₂ model and 175 for USPSTF2021 criteria.

TABLE 12
Number of persons who would need to be screened with
3MetP + 4MP + PLCOₘ₂₀₁₂ and the 4MP + PLCOₘ₂₀₁₂
model to detect one lung cancer case if the tests were applied
to the ESIA 10+ PY population at a 1%/6-year risk threshold.

≥1.0% Risk Threshold

| | | Combined |
|---|---|---|
| | Combined 4MP + | 3MetP + 4MP + |
| Strata | PLCOₘ₂₀₁₂ Model | PLCOₘ₂₀₁₂ Model |
| All | 135 | 120 |
| Low^{h} | 207 | 200 |
| Medium | 176 | 139 |
| Low + Medium | 184 | 152 |
| High | 123 | 116 |

^{h}Quantities reported for low, medium, and high-risk strata are estimated from the specimen set. Quantities reported for the combined strata (Total) are weighted to reflect the distribution of low, medium, and high-risk participants.

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this invention, and without departing from the spirit and scope thereof, can make various changes and modifications of the invention to adapt it to various usages and conditions.

What is claimed is:

1. A method of treatment of lung cancer in a patient having elevated levels of, or an elevated risk score or positive risk profile based on the patient's measured levels of, diacetylspermine (DAS), arginine, and creatine riboside, and optionally, elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1), and optionally, an elevated PLCOₘ₂₀₁₂ model score, wherein the elevated levels, risk score, positive risk profile, and/or model score classify the patient as having lung cancer, comprising administering a therapeutically effective amount of a treatment for lung cancer to the patient.

2. A method of treatment of lung cancer, comprising:
(a) identifying a patient having elevated levels of, or an elevated risk score or positive risk profile based on the patient's measured levels of, diacetylspermine (DAS), arginine, and creatine riboside, and optionally, elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1), and optionally, an elevated PLCOₘ₂₀₁₂ model score, wherein the elevated levels, risk score, positive risk profile, and/or model score classify the patient as having lung cancer; and
(b) administering a therapeutically effective amount of a treatment for lung cancer to the patient.

3. A method of determining the risk of a subject for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the subject;
(b) optionally, calculating the PLCOₘ₂₀₁₂ model score of the subject; and
(c) classifying the subject as being at risk for lung cancer or not being at risk for lung cancer based on the measured levels or based on the measured levels and the model score.

4. A method of producing a risk profile of a subject for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the subject;
(b) optionally, calculating the PLCOₘ₂₀₁₂ model score of the subject; and
(c) classifying the subject as being at risk for lung cancer or not being at risk for lung cancer based on the measured levels or based on the measured levels and the model score.

5. A method of risk stratification for a patient at risk for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1 in a biological sample obtained from the patient;
(b) optionally, calculating the PLCOₘ₂₀₁₂ model score of the patient; and
(c) determining, by processor circuitry, the risk score for the patient, wherein the risk score is determined via a scoring function derived from metabolite profiles for biological samples, and optionally, PLCOₘ₂₀₁₂ model scores, taken from a plurality of individuals that were monitored for lung cancer.

6. A method for calculating a patient's biomarker scores or risk score for lung cancer, comprising:
(a) measuring the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), in a biological sample obtained from the patient;
(b) optionally, calculating the PLCOₘ₂₀₁₂ model score of the patient; and
(c) calculating the biomarker score or risk score using the numerical values of the measured levels, and optionally, the PLCOₘ₂₀₁₂ model score, in a logistic regression model.

7. The method of either claim 5 or 6, wherein the biomarker scores or risk score for lung cancer are calculated with the equation: 0.420*[L-arginine]+0.383*[diacetylspermine]+0.184*[creatine riboside].

8. The method of any one of claims 1-6, further comprising calculating the PLCOₘ₂₀₁₂ model score.

9. The method of any one of claims 1-8, further comprising measuring the levels of or identifying a patient with elevated levels of, or an elevated risk score or positive risk profile based on the patient's measured levels of, pro-surfactant protein B (pro-SFTPB), Mucin 16 (CA125), carcinoembryonic antigen (CEA), and cytokeratin-19 fragment (CYFRA21-1).

10. The method of any one of claims 1-9, wherein calculating the PLCOₘ₂₀₁₂ model score comprises measuring a patient's age, ethnicity, educational level, body mass index (BMI), chronic obstructive pulmonary disease (COPD) status, personal history of cancer, family history of lung cancer, smoking status, smoking intensity, duration of smoking history, and duration after smoking cessation (i.e., quit time) and using the values to calculate a score with the PLCOₘ₂₀₁₂ logistic regression model.

11. The method of any one of claims 1-9, wherein the levels of pro-SFTPB, CA125, CEA, and CYFRA21-1 are determined by an immunoassay.

12. The method of claim 10, wherein the measurements of age, ethnicity, educational level, body-mass index (BMI), chronic obstructive pulmonary disease (COPD) status, history of cancer, family history of lung cancer, smoking status, smoking intensity, duration of smoking history, and duration of smoking cessation (i.e., quit time) are determined by a patient survey.

13. The method of claim 12, wherein a combined model score is calculated using the equation 0.8034*(0.420*[L-arginine]+0.383*[diacetylspermine]+0.184*[creatine riboside])+1.4238*(0.4730*[CA125]+06531*[CEA]+0.2612*[CYFRA21-1]+0.9238*[pro-SFTPB])+0.95*(PLCOₘ₂₀₁₂ model score).

14. The method of any one of claims 1-13, wherein the lung cancer is early stage (e.g., stage I or II).

15. The method of any one of claims 1-13, wherein the lung cancer is advanced (e.g., stage III or IV).

16. The method of any one of claims 1-13, wherein the levels of diacetylspermine (DAS), arginine, and creatine riboside, and optionally, pro-surfactant protein B (pro-SFTPB), Mucin 16 (CA125), carcinoembryonic antigen (CEA), and cytokeratin-19 fragment (CYFRA21-1), are elevated relative to a reference patient or group that does not have lung cancer.

17. The method of any one of claims 1-13, wherein the subject has a smoking history of ≥20 pack years.

18. The method of claim 17, wherein the subject is between the age of 50 and 80.

19. The method of any preceding claim, wherein each of diacetylspermine (DAS), arginine, and creatine riboside, and optionally, pro-surfactant protein B (pro-SFTPB), Mucin 16 (CA125), carcinoembryonic antigen (CEA), and cytokeratin-19 fragment (CYFRA21-1), generates a detectable signal.

20. The method of claim 19, wherein the detectable signals are detectable by a spectrometric method.

21. The method of claim 20, wherein the spectrometric method is chosen from UV-visible spectroscopy, mass spectroscopy, nuclear magnetic resonance (NMR) spectroscopy, proton NMR spectroscopy, nuclear magnetic resonance (NMR) spectrometry, gas chromatography, mass spectrometry (GC-MS), liquid chromatography-mass spectrometry (LC-MS), correlation spectroscopy (COSY), nuclear Overhauser effect spectroscopy (NOESY), rotating-frame nuclear Overhauser effect spectroscopy (ROESY), time-of-flight LC-MS (LC-TOF-MS), liquid chromatography-tandem mass spectrometry (LC-MS/MS), and capillary electrophoresis-mass spectrometry.

22. The method of claim 21, wherein the spectrometric method is mass spectrometry.

23. The method of claim 22, wherein the mass spectrometry is LC-TOF-MS.

24. The method of claim 1, 2, 14, or 15, wherein the treatment is chosen from surgery, chemotherapy, immunotherapy, radiation therapy, targeted therapy, or a combination thereof.

25. The method of any one of claims 1-13, wherein the calculated biomarker score, risk score, model score, or risk profile is based on sensitivity and specificity values that correspond to the risk of the subject for lung cancer.

26. The method of claim 25, wherein the sensitivity and specificity values do not differ substantially from the curve in FIG. 1 or FIG. 2.

27. The method of claim 26, wherein the sensitivity and specificity values differ by less than 10%.

28. The method of claim 27, wherein the sensitivity and specificity values differ by less than 5%.

29. The method of claim 28, wherein the sensitivity and specificity values differ by less than 1%.

30. The method of any one of claims 1-13, wherein the cutoff value comprises an AUC (95% CI) of at least 0.68.

31. The method as recited in any previous claim, further comprising assigning the patient to an appropriate risk group based on the calculated risk score.

32. The method of claim 31, wherein there are at least two risk groups.

33. The method of any one of claims 1-13, wherein the AUC of the method is greater than the AUC for a different biomarker, biomarkers, panel, assay, algorithm, model, or any combination thereof.

34. The method of claim 33, wherein the AUC is greater than 0.84.

35. The method of claim 34, wherein the AUC is between 0.84 and 0.89.

36. The method of claim 35, wherein the AUC is about 0.87.

37. The method of any one of claims 1-13, wherein the sensitivity and specificity values at a ≥1.0%/6-year risk threshold of the method are greater than the sensitivity and specificity values for a different biomarker, biomarkers, panel, assay, algorithm, model or any combination thereof.

38. The method of claim 37, wherein the sensitivity is greater than 0.88 and the specificity is greater than 0.56.

39. The method of claim 38, wherein the sensitivity is between 0.88 and 0.90 and the specificity is between 0.56 and 0.60.

40. The method of claim 39, wherein the sensitivity is about 0.90 and the specificity is about 0.60.

41. The method of any one of claims 33-40, wherein the model is PLCOₘ₂₀₁₂ alone.

42. The method of any one of claims 33-40, wherein the biomarkers are pro-SFTPB, CA125, CEA, and CYFRA21-1 alone.

43. The method of any one of claims 33-40, wherein the model is PLCOₘ₂₀₁₂ and the biomarkers are pro-SFTPB, CA125, CEA, and CYFRA21-1.

44. The method of any one of claims 41-43, wherein the cutoff points of the respective methods are used for classification.

45. The method of any one of claims 41-43, analyzed by the same statistical methods.

46. The method as recited in any previous claim, wherein the levels of diacetylspermine (DAS), arginine, and creatine riboside, and optionally, the levels of pro-surfactant protein B (pro-SFTPB), Mucin 16 (CA125), carcinoembryonic antigen (CEA), and cytokeratin-19 fragment (CYFRA21-1), are measured against a given threshold value or values.

47. The method of claim 46, wherein the values exceed the threshold value or values and the patient is classified as being at risk for lung cancer.

48. The method of claim 46, wherein the values are below the threshold value or values and the patient is classified as being not at risk for lung cancer.

49. The method of claim 48, wherein the patient is subsequently designated for further lung cancer screening or treatment.

50. The method of claim 49, wherein the screening is chosen from endoscopic ultrasound, magnetic resonance imaging (MRI), and computed topography (CT) scans.

51. The method of claim 50, wherein the screening is performed annually.

52. The method of claim 50, wherein the screening is performed semi-annually.
